(19) United States
(12) Patent Application Publication
(10) Pub. No.: US 2022/0327954 A1
(43) Pub. Date: Oct. 13, 2022

(54) EXPERT-DRIVEN, TECHNOLOGY-FACILITATED INTERVENTION SYSTEM FOR IMPROVING INTERPERSONAL RELATIONSHIPS

(71) Applicants: Gayla MARGOLIN, Los Angeles, CA (US); Adela C. AHLE, San Francisco, CA (US); Matthew William AHLE, San Francisco, CA (US); Theodora CHASPARI, College Station, CA (US); Shrikanth Sambasivan NARAYANAN, Santa Monica, CA (US)

(72) Inventors: Gayla MARGOLIN, Los Angeles, CA (US); Adela C. AHLE, San Francisco, CA (US); Matthew William AHLE, San Francisco, CA (US); Theodora CHASPARI, College Station, CA (US); Shrikanth Sambasivan NARAYANAN, Santa Monica, CA (US)

(73) Assignee: University of Southern California, Los Angeles, CA (US)

(21) Appl. No.: 17/849,996

(22) Filed: Jun. 27, 2022

Related U.S. Application Data
(63) Continuation-in-part of application No. 16/291,399, filed on Mar. 4, 2019.
Application No. 16/291,399 is a continuation-in-part of application No. 16/501,128, filed on Mar. 2, 2018, now abandoned.
(60) Provisional application No. 62/563,192, filed on Sep. 26, 2017.

(51) Int. Cl. G09B 19/00

(52) U.S. Cl. CPC G09B 19/00

(57) ABSTRACT

A method for promoting interpersonal interactions includes a step of receiving data streams from a plurality of mobile smart devices from a plurality of users, the data streams recording information about users' daily lives. Intervention signals are sent to a user in response to data acquired from two or more individuals and interpreted with respect to user internal states, moods, emotions, predetermined behaviors, and interactions with other users.

### CROSS-REFERENCE TO RELATED APPLICATIONS

This application is a continuation-in-part of U.S. patent application Ser. No. 16/291,399 filed Mar. 4, 2019, and is a continuation-in-part of U.S. patent application Ser. No. 16/501,128 filed Mar. 2, 2018, which claims the benefit of U.S. provisional application Ser. No. 62/563,192 filed Sep. 26, 2017, the disclosures of which are hereby incorporated in their entirety by reference herein.

### STATEMENT REGARDING FEDERALLY SPONSORED RESEARCH OR DEVELOPMENT

The invention was made with Government support under Contract No. R21 HD072170-A1 awarded by the National Institutes of Health/National Institute of Child Health and Human Development; Contract Nos. BCS-1627272, DGE-0937362, and CCF-1029373 awarded by the National Science Foundation; and Contract No. UL1TR000130 awarded by the National Institutes of Health. The Government has certain rights to the invention.

### TECHNICAL FIELD

In at least one aspect, the present invention provides a novel, automatic framework for the development and evaluation of mobile, adaptive interventions used to improve interpersonal relationships. In particular, through the integration of expert-knowledge and automated, data-driven methods, this technology-facilitated framework monitors, measures, and quantifies signal-derived human information and provides prompts, suggestions, and support to elicit behavioral change.

### BACKGROUND

Interpersonal relationships refer to acquaintances, close bonds, and affiliations between two or more people across personal, business, educational, and social domains. The quality of these interpersonal relationships is crucial for people's quality of life, well-being, and health. Strained personal and family relationships have been extensively linked to a variety of negative outcomes, including psychological disorders and physical health problems across the lifespan (Burman & Margolin, 1992; Coan, Schaefer, & Davidson, 2006; Grewen, Andersen, Girdler, & Light, 2003; Springer, Sheridan, Kuo, & Carnes, 2007; Holt-Lunstad, Smith, & Layton, 2010; Leach, Butterworth, Olesen, & Mackinnon, 2013; Robles & Kiecolt Glaser, 2003). Similarly, problems in professional relationships have been associated with reduced productivity and decreased well-being (Lawler, 2010; Sacker, 2013; Sonnentag, Unger, & Nagel, 2013).

Current interventions aiming to improve relationship functioning largely rely on participants' retrospective self-reports of their relationship functioning and therapists' observations of their interaction quality. While these are valuable sources of information, traditional therapy interventions have shown only moderate effectiveness in clinical trials (e.g., Lunbald & Hansson, 2005); treatment efficacy may in part be limited by the inherently subjective nature of human judgment; moreover, these interventions cannot provide in-the-moment feedback when problems actually occur in people's day-to-day lives. Additionally, traditional therapies reach only a fraction of individuals who are experiencing significant relationship problems and related difficulties (Mayberry, Nicewander, Qin, & Ballard, 2006). Emerging technological advances now make it possible to monitor people outside the laboratory and collect real-life data about their behavior, interactions, and mental state, and felt-sense. The valuable information about interpersonal dynamics embedded in this multimodal data is thus useful for creating novel, automated and semi-automated intervention systems tailored to individuals to improve their relationships. Such intervention systems rely on human knowledge provided by life-sciences experts accompanied by data-scientific solutions that are able to enhance and complement the human-guided suggestions. In this way, technology can increase people's awareness of emotions, feelings, and problematic behaviors when they occur, provide warnings before problems or conflicts develop, and identify positive and negative interpersonally-relevant states and events beyond what can be identified through traditional therapy. Therapists, on the other hand, can obtain quantitative feedback on their clients' behavior and progress and can adjust interventions with data-driven solutions. These techniques could, therefore, improve individual mental and physical health, democratize access to mental health care, and contribute to saved revenue over time.

Beyond traditional office-based therapy, current online interventions widely rely on web-based educational materials and questionnaires to improve and support interpersonal relationships (Doss, Bensen, Georgia, & Christensen, 2013; Larson et al., 2007). These strategies are widely accessible and can provide initial feedback on relationship quality, but are not highly detailed, do not provide in-the-moment monitoring, feedback, and intervention, and cannot be easily personalized. Other interventions involve remote, online conference sessions with experts (Ianakieva et al., 2016). While these can be effective, they are impossible to scale in large and underprivileged populations, since the presence of experts is costly and not always guaranteed. One potential avenue to increase access to and the effectiveness of interpersonal interventions is to use ambulatory technology that can understand people's behavior, emotions, and felt-sense and provide automated suggestions for positive changes. Recent interdisciplinary studies have examined the possibility of real-life ambulatory monitoring to capture well-being indices and track the progress of mental health conditions and corresponding therapies (Hung, & Englebienne, 2013; Lane et al., 2014; Gideon et al., 2016). However, these studies have focused solely on individual-level functioning, with no previous work, to the best of our knowledge, attempting to monitor and improve social dynamics and interpersonal relations in groups of people.

Accordingly, there is a need for improved methods and systems for monitoring and improving interpersonal relationships.

### SUMMARY

The present invention solves one or more problems of the prior art by providing in at least one embodiment, a system that involves the development, tracking, and evaluation of data-driven interventions through a technology-support system that integrates prior knowledge of human-experts, processes multimodal information acquired from a group of people, and uses data-science, machine learning, and automatic control-based methodologies to create individualized suggestions for altering daily patterns and dynamics of interpersonal relationships (e.g., predict and prevent conflict episodes, increase the frequency of positive interactions, support relationship bonding, aid in expressing viewpoints or emotions in an adaptive manner, effectively problem-solve relationship issues, improve conflict resolution strategies, resolve conflict or restore relationship functioning after conflict has occurred). This system has applications for a variety of relationships (e.g. couples, friends, families, co-workers) and can be employed by individuals or implemented on a broad scale by institutions and large interpersonal networks (e.g. hospitals, military settings).

### BRIEF DESCRIPTION OF THE DRAWINGS

FIG. 1 is a schematic of a framework implementing embodiments of the invention.

FIG. 2 is a schematic of a smart device used in the system of FIG. 1.

FIG. 3. A model for interpersonal dynamics that occur when a set of individuals linked through a relationship interacts with each other and with their environment.

FIG. 4A is a schematic representation of a classification system. Multimodal classification (task 3) between conflict and non-conflict samples (S1 . . . SN) used combinations of features from each modality: self-reported mood and quality of interactions (MQI), electrodermal activity (EDA), electrocardiogram (ECG) activity, EDA synchrony measures, language, and acoustic information.

FIG. 4B is an example of a decision tree.

FIG. 5. Schematic illustrating a method in which personal interactions are evaluated and classified by considering clusters of users with similar traits.

FIG. 6. Receiver operating characteristic (ROC) curves for self-reported mood and quality of interaction (such as stressed, happy, sad, nervous, angry, and close) and multimodal feature groups. Multimodal-F-a=female EDA, psychological, personal, interaction, context; Multimodal-F-b=female self-reported MQI, EDA, psychological, personal, acoustic, interaction, context; Multimodal-M-a=male ECG activity, psychological, Acoustic, interaction, context; Multimodal-M-b=male self-reported MQI, EDA synchrony, paralinguistic, personal, acoustic, interaction, context.

### DETAILED DESCRIPTION

Reference will now be made in detail to presently preferred compositions, embodiments and methods of the present invention, which constitute the best modes of practicing the invention presently known to the inventors. The Figures are not necessarily to scale. However, it is to be understood that the disclosed embodiments are merely exemplary of the invention that may be embodied in various and alternative forms. Therefore, specific details disclosed herein are not to be interpreted as limiting, but merely as a representative basis for any aspect of the invention and/or as a representative basis for teaching one skilled in the art to variously employ the present invention.

It is also to be understood that this invention is not limited to the specific embodiments and methods described below, as specific components and/or conditions may, of course, vary. Furthermore, the terminology used herein is used only for the purpose of describing particular embodiments of the present invention and is not intended to be limiting in any way.

It must also be noted that, as used in the specification and the appended claims, the singular form “a,” “an,” and “the” comprise plural referents unless the context clearly indicates otherwise. For example, reference to a component in the singular is intended to comprise a plurality of components.

The term “comprising” is synonymous with “including,” “having,” “containing,” or “characterized by.” These terms are inclusive and open-ended and do not exclude additional, unrecited elements or method steps.

The phrase “consisting of” excludes any element, step, or ingredient not specified in the claim. When this phrase appears in a clause of the body of a claim, rather than immediately following the preamble, it limits only the element set forth in that clause; other elements are not excluded from the claim as a whole.

The phrase “consisting essentially of” limits the scope of a claim to the specified materials or steps, plus those that do not materially affect the basic and novel characteristic(s) of the claimed subject matter.

With respect to the terms “comprising,” “consisting of,” and “consisting essentially of,” where one of these three terms is used herein, the presently disclosed and claimed subject matter can include the use of either of the other two terms.

Throughout this application, where publications are referenced, the disclosures of these publications in their entireties are hereby incorporated by reference into this application to more fully describe the state of the art to which this invention pertains.

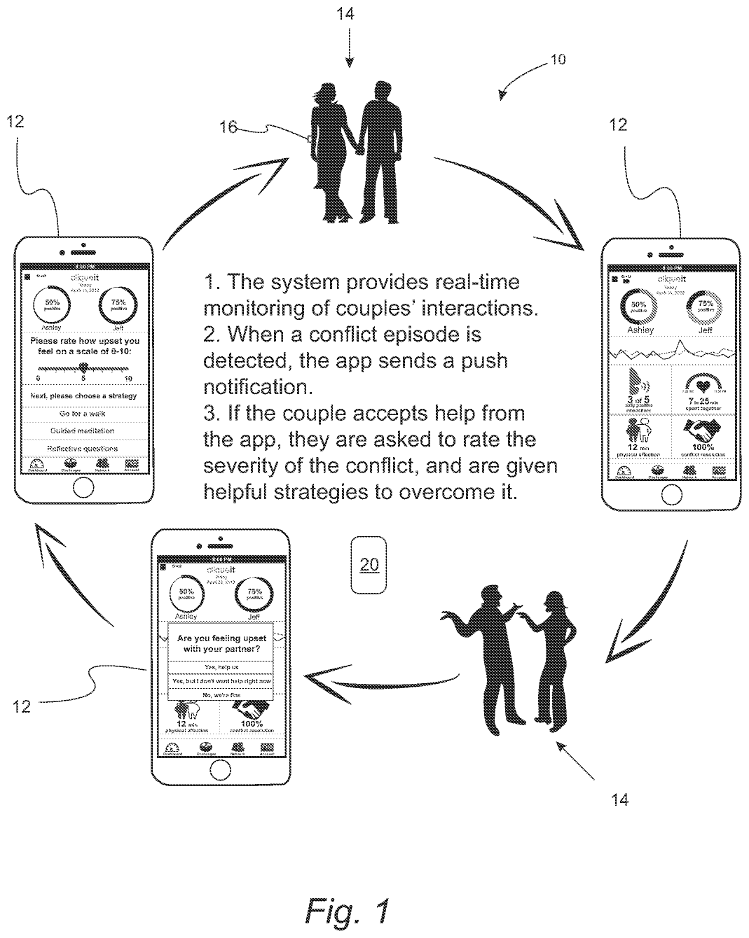

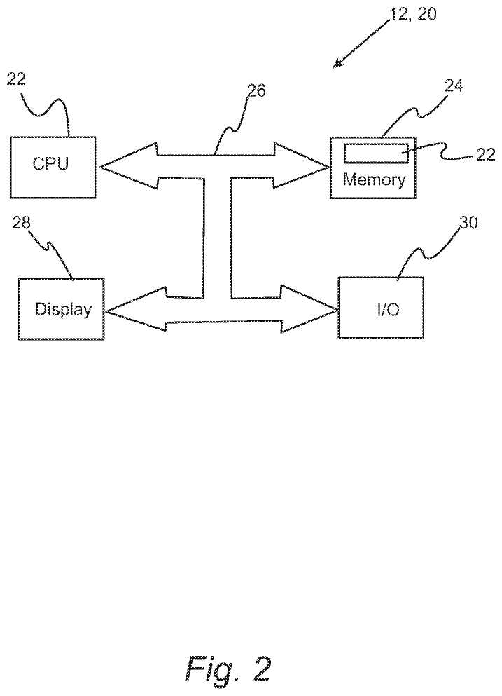

In an embodiment, a method for monitoring interpersonal relationships is provided. The method includes a step of receiving data streams from a plurality of smart devices from a plurality of users. The data streams record information about users' daily lives. Typically, the data streams are obtained directly from the smart devices or from sensors worn by the plurality of users in communication (e.g., wireless or wired) with the smart devices. Typically, the smart devices are mobile smart devices. Examples of smart devices include, but are not limited to, smart phones (e.g., iPhones), tablet computers (e.g., iPads), and the like. Intervention signals are sent to a user in response to data acquired from two or more individuals and interpreted with respect to user internal states, moods, emotions, predetermined behaviors, and interactions with other users. In one variation, the method can be implemented within a smart device in the possession of a user. An app on the smart device can provide real-time monitoring of interactions by receiving the data streams from the user in possession of the device and from other users. If intervention is required, the app can provide a message providing such intervention. For example, when a conflict episode is detected, the app can send a push notification. If the uses accepts help from the app, they are asked to rate the severity of the conflict, and are given helpful strategies to overcome it. In another variation, the data streams are received by and/or the interventions signals generated by a monitor smart device. FIGS. 1, 2 which are described below in more detail provide illustrations of systems that implement the present method of monitoring and understanding interpersonal relationships.

In a variation, the intervention signals are determined by algorithmic signal processing and/or machine learning methods such that the intervention signals are responsive, interactive, and adaptive to the users. For example, multimodal data streams can be obtained on a variety of metrics, such as physiological arousal, speech, vocal pitch and tone, GPS location, etc. These data are inputted machine learning algorithms (e.g., neural networks, support vector machines) used to detect interpersonal states of interest (e.g., conflict, feelings of closeness). Ground truth for states of interest are determined via self-report from phone surveys and observational data from audio and/or video collected. Algorithms that are developed (and evaluated via standard evaluation metrics, e.g., kappa, balanced accuracy) are applied. When a state of interest is detected, intervention prompts that are specific to each individual will be sent (e.g., if conflict is predicted, a warning that conflict might occur will be sent and emotion regulation exercise intervention might be suggested). Interventions can focus on increasing awareness of mood or relationship states and use positive reinforcement principles, might assist in helping regulate emotions, or may attempt to warn or predict conflict. Prompts are interactive and adaptive to users because they are sent in moments of need for that individual, based upon what is detected at that point in time. In one particular refinement, signal-derived features are extracted from the data streams. The signal derived features provide inputs to a trained neural network that detect or predict interpersonal classifications that allow selection of a predetermined intervention to be sent.

In a variation, the method further includes a step of incorporating human expert knowledge into a determination of the intervention signals. For example, a therapist may decide to focus on a particular goal (e.g., increasing the amount of time spent together and interacting). The therapist could set the software to send reminder prompts for specific goals according to his or her own theory and practice-based knowledge of what might be helpful. The human expert knowledge is integrated and includes prompts sent at random intervals and/or according to specific time schedules. In a refinement, reminders designed to help users reach their daily goals can be sent. The reminders can include spending a certain amount of time together, achieving a certain ratio of positive to negative interactions, or having a certain amount of physical contact.

In another variation, the sending of interventions can be triggered by algorithms that automatically detect and predict moods and events to send prompts to oneself or to other users in a social network. For example, if the algorithms detect that conflict is likely, the intervention system could be programmed to send a prompt that says “You are at risk for having conflict with your child/husband/friend. Would you like to try a relaxation exercise?” Users would then be guided through a computer-assisted relaxation module. Prompts could also be sent cross-person in the network. For example, when increases in stress of 40% above baseline are observed, a prompt could be sent to a family member that say “Your husband/child/friend is feeling more stressed today than usual.” The interventions can also include sending prompts after events of interest have occurred. The moods and events can include risky behaviors, extreme emotions, and/or negative moods. Further, the prompts can include warning people that conflict or other events are likely to occur, prompting people to engage in relaxation exercises, take a break, give a compliment, or to do something nice for someone else. In a refinement, the prompts can instruct users to reflect on an occurrence of an event, engage in relationship building activities, initiate positive contact, or discuss a topic together.

In another variation, the method includes a step of providing feedback to the users to encourage beneficial aspects of interpersonal relationships. To provide feedback, expert-knowledge can be applied with personal and interpersonal information captured from human monitoring systems integrated through signal processing, data-scientific, and machine learning solutions. Further, a human state can be recognized, understood, and predicted from this information and actionable feedback can provided to improve it in relation to corresponding relationship functioning. Measurable indices of individual and interpersonal behavior consisting of input for closed-loop systems can automatically provide suggestions towards a desired state. For example, the system would identify a state of interest (e.g., low mood). When this human state is recognized and/or predicted, the system sends feedback to the user and provides intervention prompts designed to improve behavior or functioning (e.g., improve mood). Examples of feedback provided to the user and possible interventions to be sent are provided below in Table 1 (including the target state to intervene upon, the general goal to initiated, and the prompt that could be sent).

TABLE 1

| Target | Amplifying attachment bonds |
|---|---|
| Closeness | You are feeling closer with your [child/partner] compared to |
| | [an hour ago/yesterday/last week]. |
| Positivity | You have had more positive interactions with your |
| | [child/partner] in the past [hour/day/week]. |
| Time | You have spent more quality time with your [child/partner] in |
| | the past [hour/day/week]. |

| Target | Increasing emotional awareness |
|---|---|
| Happiness | [You have/your child has] been feeling happier in the past |
| | [hour/day/week]. |
| Sadness | [You have/your child has] been feeling sadder in the past |
| | [hour/day/week]. |
| Anxiety | [You have/your child has] been feeling more anxious in the |
| | past [hour/day/week]. |
| Anger | [You have/your child has] been feeling angrier in the past |
| | [hour/day/week]. |
| Stress | [You have/your child has] been feeling more stress in the |
| | past [hour/day/week]. |

| Option | Improving emotion regulation skills |
|---|---|
| 1 | Mindfulness meditation |
| 2 | Guided deep breathing |
| 3 | Progressive muscle relaxation |
| 4 | Challenging unhelpful thoughts |
| 5 | Noticing and accepting emotions |

In yet another variation, heuristic, machine-learning, or control-theoretical approaches are applied and can be automatically trained, tuned, and/or perturbed towards optimizing a desired criterion to minimize conflict and maximize positive interactions. For example, an algorithm might predict a certain target state, such as conflict and might send a certain intervention, such as a mindfulness meditation. The system would then continue to monitor to determine whether a conflict occurred. At other instances, other interventions might be prompted, such as a guided deep breathing exercise. At these instances, the system would continue to monitor to determine whether a conflict occurred. The result, i.e., whether the conflict occurred or not, would then provide data in the algorithm and be programmed for future prompts. The prompts that were most effective for that person would be administered more frequently while those prompts that were least effective for that person would be administered less frequently. In this way, we would provide the most effective interventions to maximize treatment gains. This would be specific to each individual. This could be accomplished via a variety of models, such as reinforcement learning and a K-armed bandit. The goal is to find the optimal prompt for a participant. The K-armed bandit algorithm will observe the participant, the environment, and previously administered prompts, and will provide a new prompt associated with a reward. Based on this and the updated state space, the algorithm will renew its policy and provide a revised prompt. In this way, the algorithm will learn and adapt by observing the result of an action. The state space for participant at a given time will be represented by 1) signal-based features of the target participant, 2) signal-based features of his/her partner (if they are interacting), and 3) feedback attributes to avoid continuously providing the same message. The reward policy will include the participant's retrospective ratings of the perceived effectiveness of the administered prompt.

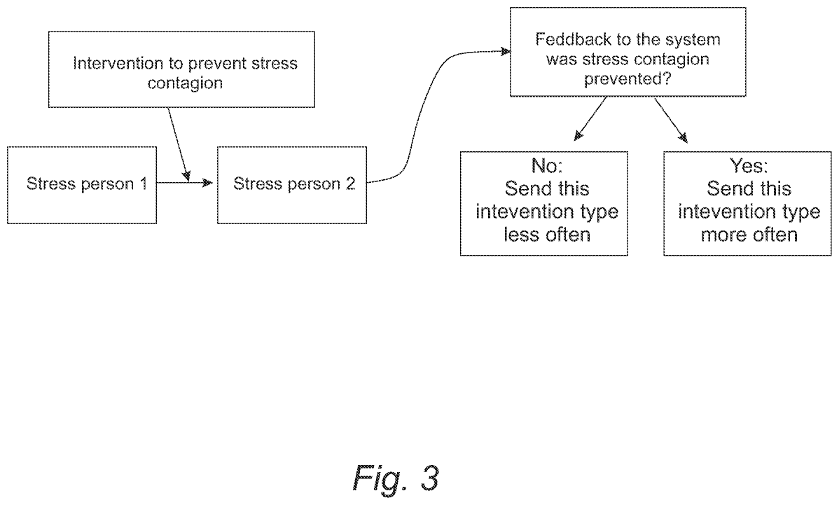

A model can be constructed for interpersonal dynamics that occur when a set of individuals linked through a relationship interacts with each other and with their environment. For example, these prompts and interventions could be sent to intervene in interpersonal functioning or processes that occur across people, e.g., covariation in mood states where one person mitigates stress in another person, stress contagion processes, conflict escalation, feelings of closeness between partners (beyond individual stress states). The model uses machine learning and reinforcement methods as described above and would incorporate interpersonal elements. (see, FIG. 3). The method can further include a step of learning each other's patterns over time so that accuracy and effectiveness of interventions increase with use.

In still another variation, the method includes a step of investigating an impact of each prompt and intervention on individual and interpersonal functioning and providing feedback about which interventions are most helpful population-wide and which are better for specific users, couples, or groups of users. This could be done via a combination of machine learning/reinforcement learning models to see which interventions contribute most to changes in desired outcomes, clustering analyses and the development of sub-population specific models, as well as other statistical methods such as multilevel modeling and structural equation modeling to determine which interventions impact treatment effects for which individuals or groups of people. The intervention schemes can be performed quantitatively through signal- and data-derived measures indicative of individual characteristics and relationship functioning concepts. This means that intervention sets or designs may be determined from the machine-learning identified interventions that are found to be most useful (i.e., the exact structure of the intervention frequency, type, etc. would be determined from algorithms conducting ongoing monitoring of whether it was effective for that individual or group of individuals). Moreover, feedback regarding which interventions are most helpful to which people could be provided directly through the intervention system (e.g., a prompt sent through phones telling a person that this exercise or technique is particularly helpful for you) or through therapist-provided feedback. The therapist, for example, might review the intervention system data and determine that a particular intervention is most effective based on the remote monitoring data he or she has reviewed. This information could also then be used to provide feedback to the client and to design future intervention schemes. For example, if a particular intervention is determined to be effective for a group of people, e.g., females, then the frequency with which that intervention is sent could be updated for that group of people.

In a variation, data streams are classified or quantified into classifications or quantifications. In a refinement, these classifications can be used to determine the interventions signals that are sent. Details regarding classification and quantification are describe in more detail in co-pending patent application Ser. No. 16/291,225 filed Mar. 4, 2019, the entire disclosure of which is hereby incorporated by reference. In this regard, the method can further include a step of computing signal-derived features from the data streams. Acoustic features include motor timing parameters of speech production (e.g., speaking rate and pause time), prosody and intonation (e.g., loudness and pitch), and frequency modulation (e.g., spectral coefficients). Linguistic features include word count, frequency of parts of speech (e.g., nouns, personal pronouns, adjectives, verbs), frequency of words related to affect, stress, mood, family, aggression, work. Physiological measures include skin conductance level, mean skin conductance response frequency and amplitude, rise and recovery time of skin conductance responses, average, standard deviation, minimum, and maximum of the inter-beat interval (IBI), average beats per minute, heart rate variability, R-R interval, as well as the very-low (<0.04 Hz), low (0.04-0.15 Hz), and high (0.15-0.4 Hz) frequency component of the MI, breathing rate, breathing rate variability, mean acceleration, acceleration entropy, and/or Fourier coefficients of acceleration. Similarity measures between the two partners can be computed with respect to the above features in order to integrate momentary co-regulation as a feature to the machine learning system. In addition to that, raw signals could be used as features (e.g., inputs) for the machine learnings algorithms, which will learn feature transformations for the outcome of interest. Custom made toolboxes available online and developed in the lab can be used to derive such metrics. The signal-derived representation can be computed by knowledge-based design and/or data-driven analyses, which can include clustering. For example, all of these data (raw signal and extracted measures) can be used as features for algorithm development where the ground truth is established via either or a combination of self-report data from concurrent phone surveys or through observational codes obtained from audio and/or video data. Ground truth constructs (e.g., conflicts) can then be used as labels in algorithms to detect the identified target states for monitoring. In the context of the present invention, the term “algorithm” includes any computer-implemented method that is used to perform the methods of the invention. In particular, machine learning algorithms include a variety of models, such as neural networks, support vector machines, binary decision trees, and the like. Leave-one-out cross validation would be conducted to assess accuracy. Standard evaluation metrics can be applied (e.g., kappa, F1-score, mean absolute errors). In addition to supervised learning methods, unsupervised methods could be used to detect clusters in the data that were not hypothesized beforehand (e.g., supervised and unsupervised neural networks). Theory-based models could include using questionnaire or other data to build subpopulation specific models for increased accuracy (e.g., subpopulation models built on aggression levels).

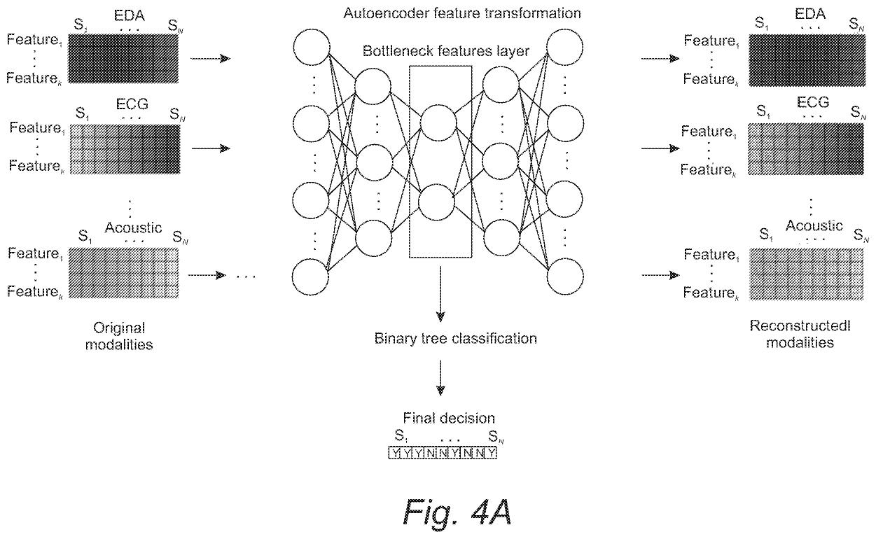

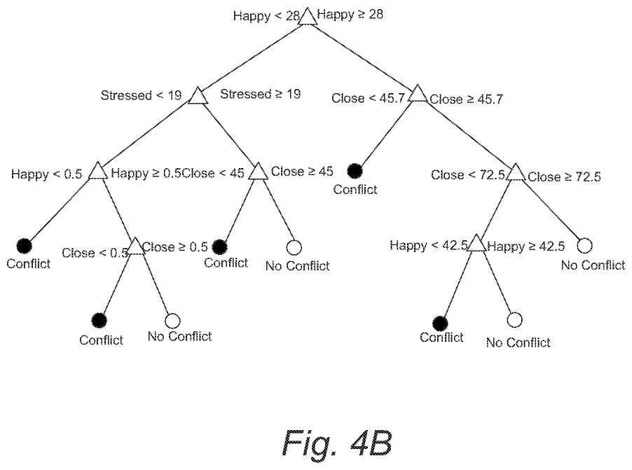

In a refinement, the signal-derived features (e.g., signal derived representations) are used as inputs for machine learning, data mining, and statistical algorithms that can be used to determine what factors, or combinations of factors, predict a clarification or a variety or relationship dimensions, such as conflict, relationship quality, or positive interactions. This means use machine learning to detect all the state constructs of interest via a variety of algorithms (e.g., neural networks, support vector machine, and the like). Furthermore, one can obtain data on relationship functioning over time (e.g., from questionnaire data or from the phone-based metrics) to determine which families or couples (or other systems of people) are experiencing increases or decreases in relationship functioning (e.g., decreases in conflict). Machine learning methods can then be used to retroactively determine what features or combinations thereof predict changes in relationship functioning over time. FIG. 4A provides an example of a procedure for training an algorithm. A combination of self-report data, coding of interviews, observations, videos, and/or audio recordings can be compared to the signal-derived features to determine the accuracy of the systems. In FIG. 4A, an autoencoder neural network is trained with features (e.g., feature₁ . . . featureₖ) as input modalities characterizing each of a variety of signals (e.g., EDA signals, ECG signals, Acoustic signals, and the like). The objective of the autoencoder is to learn a reduced representation of the original feature set. The reduced feature set is obtained from the bottleneck layer, which is learned so that the right side of the autoencoder network can generate a representation as close as possible to the original signal from the reduced encoding. From the bottleneck layer, inputs for drafting a binary decision tree are extracted. The output at the right of FIG. 4A provide reconstructed modalities which should provide a good approximation of the inputs if the neural network has been properly trained. Once trained, the neural network and the decision tree can be used to perform the classification. FIG. 4B is an example of a decision tree. In a variation, statistics, e.g., regression analyses, latent class analysis can be used to predict changes in relationship functioning.

In another variation, individual models are used to increase classification accuracy since patterns of interaction may be specific to individuals, couples, or groups of individuals. This could involve building sub-population specific machine learning models. Models would leverage common information across all people and then fine-tune decisions based on the sub-population (e.g., level of aggression in the relationship, level of depression, sociodemographic factors) of interest. Decisions will be made for clusters of people with common characteristics, improving accuracy and reducing the amount of data needed for training. Models could use multi-task learning to leverage useful information from related sub-populations. By operationalizing multi-task learning as a feature-learning approach, it can be assumed that people share some general feature representations, while specific representations can later be learned for every subpopulation. Multi-task learning could be implemented using deterministic and probability methods (i.e., train the first layers of the feedforward neural networks based on the entire dataset to represent common feature embeddings and then refine the last layers for each subpopulation separately).

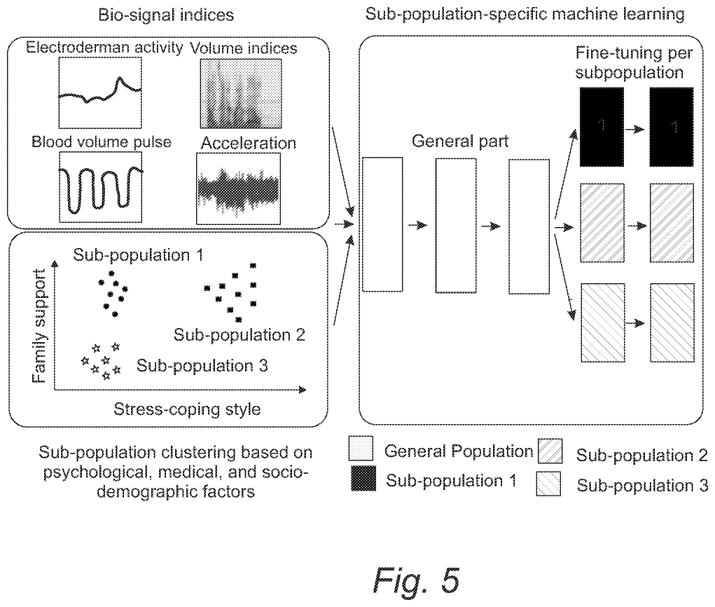

In a refinement, active and semi-supervised learning methods are applied to increase predictive power as people continue to use a system implementing the method of monitoring and understanding interpersonal relationships. For example, reinforcement learning models could be used to determine whether to administer a phone survey at a given time. The goal of the algorithm would be to solve a sequential decision problem, where at each stage there are two possible actions (to administer or not administer the phone survey). The algorithm would attempt to achieve optimal balance between maximizing a cumulative reward function (i.e., correct identifying a target state such as conflict) and exploring unseen regions of the input (i.e., administering a phone survey for exploring bio-signal patterns that have not yet been observed). The state-space of the algorithm would be represented by the bio-signals, estimates from the sub-population specific machine learning models, and time elapsed since the last phone survey (to prevent the continuous administration of phone surveys). Receiving estimates from subpopulation specific machine learning algorithms will prevent the reinforcement learning algorithms from exploring too many irrelevant bio-signal patterns in the first steps. The reward function would be the person's response to the phone survey over previous time points with similar bio-signal patterns to the current one. Similarity could be computed via a distance measure (e.g., Euclidean norm) between the current and previous bio-signal indices in past phone surveys. The current action would reflect the trade-off between maximizing the cumulative reward (i.e., observing the state) and adequately exploring unseen feedbacks (i.e., a bio-signal pattern for which a phone survey has not been received before). If the algorithm decides to administer a survey, the reward function will be populated with an additional pair of values and the cumulative reward will be updated based on the self-reported data. The goal of this algorithm (e.g., 1-armed bandit) would be to gradually learn each person's state-related bio-signals over time and to administer phone surveys only when the state is detected. FIG. 5 provides an example of a clustering algorithm in which users are grouped by similar characteristics (e.g., family support, stress coping style, and the like). A neutral network is used to provide inter-group classifications (e.g., criteria of classifications is group specific).

As set forth above, characterization of the interpersonal relations can be performed by a neural network that receives by the data collecting from the smart phones as inputs. The neural network can be trained from previously obtained data (e.g., the signal derived features set forth above) that has an assigned known classification which can be obtained by users self-reporting (e.g., stating they are in conflict). As set forth below in the examples, the neural network can be used to provide training inputs to a decision tree. The computer tree can be alternatively used (e.g., instead of using a neural network) to perform the classification step set forth above.

In an embodiment, a system that implements the previously described methods is provided. With reference to FIGS. 1 and 2, the system 10 includes a plurality of smart devices 12 operated by a plurality of users 14. In general, at least one smart device or a combination of smart devices execute steps of receiving data streams from a plurality of smart devices in the possession of a plurality of users where the data streams recording information about users' daily lives and sending intervention signals to a user in response to data acquired from two or more individuals and interpreted with respect to user internal states, moods, emotions, predetermined behaviors, and interactions with other users. As set forth, the smart devices are typically mobile smart devices (e.g., smart phone, tablets, and the like). In a variation, the system further includes a plurality of sensors 16 (e.g., heart rate sensors, blood pressure sensors, etc.) worn by the plurality of users. The plurality of mobile smart devices 12 used this system typically includes a microprocessor and a non-volatile memory on which instructions for implementing the method are stored. In a variation as depicted in FIGS. 1 and 2, system 12 provides real-time monitoring of couples' or a plurality of users' interactions. When a conflict episode is detected, the app sends a push notification. If the couple accepts help from the app, they are asked to rate the severity of the conflict, and are given helpful strategies to overcome it. In a refinement as set forth above, the data streams are received by and/or the interventions signals generated by a monitor smart device 20. Device 20 may also perform the classification. For example, smart devices 12 and monitor smart device can include computer processor 22 that executes one, several, or all of the steps of the method. It should be appreciated that virtually any type of computer processor may be used, including microprocessors, multicore processors, and the like. The steps of the method typically are stored in computer memory 24 and accessed by computer processor 22 via connection system 26. In a variation, connection system 26 includes a data bus. In a refinement, computer memory 24 includes a computer-readable medium which can be any non-transitory (e. g., tangible) medium that participates in providing data that may be read by a computer. Specific examples for computer memory 24 include, but are not limited to, random access memory (RAM), read only memory (ROM), hard drives, optical drives, removable media (e.g. compact disks (CDs), DVD, flash drives, memory cards, etc.), and the like, and combinations thereof. In another refinement, computer processor 22 receives instructions from computer memory 24 and executes these instructions, thereby performing one or more processes, including one or more of the processes described herein. Computer-executable instructions may be compiled or interpreted from computer programs created using a variety of programming languages and/or technologies including, without limitation, and either alone or in combination, Java, C, C++, C #, Fortran, Pascal, Visual Basic, Java Script, Perl, PL/SQL, etc. Display 28 is also in communication with computer processor 22 via connection system 16. Smart device 12 also optionally includes various in/out ports 30 through which data from a pointing device may be accessed by computer processor 22. Examples for monitor smart device 20 include, but are not limited to, desktop computers, smart phones, tablets, or tablet computers.

The following examples illustrate the various embodiments of the present invention. Those skilled in the art will recognize many variations that are within the spirit of the present invention and scope of the claims.

Prototype Model

Using mobile computing technology, our field study collected self-reports of mood and the quality of interactions (MQI) between partners, EDA, ECG activity, synchrony scores, language use, acoustic quality, and other relevant data (such as whether partners were together or communicating remotely) to detect conflict in young-adult dating couples in their daily lives. We conducted classification experiments with binary decision trees to retro actively detect the number of hours of couple conflict.

To assess our approach's usefulness, our study addressed four interrelated research questions that generated four tasks:

Question 1: Are theoretically driven features related to conflict episodes in daily life? Task 1: We conducted individual experiments for theoretically driven features, including self-reported MQI, EDA, ECG activity, synchrony scores, personal pronoun use, negative emotion words, certainty words, F0, and vocal intensity.

Question 2: Are unimodal feature groups related to conflict episodes in daily life? Task 2: We combined the features into unimodal groups to determine the classification accuracy of different categories of variables.

Question 3: Are multimodal feature combinations related to conflict episodes in daily life? Task 3: We combined the feature groups into multimodal indices to examine the performance of multiple sensor modalities.

Question 4: How do multimodal feature combinations compare with the couples' self-report data? Task 4: We statistically compared the classification accuracy of our multimodal indices to the couples' self-reported MQI to ascertain the potential of these methods to identify naturally occurring conflict episodes beyond what participants themselves reported, hour by hour.

Our objective here is to present preliminary data and demonstrate our classification system's potential utility for detecting complex psychological states in uncontrolled settings. Although this study collected data on dating couples, these methods could be used to study other types of relationships, such as friendships or relation-ships between parents and children.

Research Methodology

The participants in our study consisted of young-adult dating couples from the Couple Mobile Sensing Project, with a median age of 22.45 years and a standard deviation (SD) of 1.60 years. The couples were recruited from the greater Los Angeles area and had been in a relationship for an average of 25.2 months (SD=20.7). Participants were ethnically and racially diverse, with 28.9 percent identifying as Hispanic, 31.6 percent Caucasian, 13.2 percent African-American, 5.3 percent Asian, and 21.1 percent multiracial.

Out of 34 couples who provided data, 19 reported experiencing at least one conflict episode and thus were included in the classification experiments. All study procedures were approved by the USC Institutional Review Board.

Measures

All dating partners were outfitted with two ambulatory physiological monitors that collected EDA and ECG data for one day during waking hours. They also received a smartphone that alerted them to complete hourly self-reports on their general mood states and the quality of their interactions. The self-report options, which were designed to assess general emotional states relevant to couple interactions, included feeling stressed, happy, sad, nervous, angry, and close to one's partner. Responses ranged from 0 (not at all) to 100 (extremely).

Additionally, each phone continuously collected GPS coordinates, as well as 3-minute audio recordings every 12 minutes from 10:00 a.m. until the couples went to bed.

Physiological indices. We collected physiological measures continuously for one day, starting at 10:00 a.m. and ending at bedtime. EDA, activity count, and body temperature were recorded with a Q-sensor, which was attached to the inside of the wrist using a band. ECG signals were collected with an Actiwave, which was worn on the chest under the clothing. ECG measures included the interbeat interval (MI) and heart rate variability (HRV), and EDA features consisted of the skin conductance level (SCL) and the frequency of skin conductance responses (SCRs). Estimates of synchrony, or covariation in EDA signals between romantic partners, were obtained using joint-sparse representation techniques with appropriately designed EDA-specific dictionaries. (T. Chaspari et al., 2015; the entire disclosure of which is hereby incorporated by reference).

We used computer algorithms to detect artifacts, which were then visually inspected and revised. All scores were averaged across each hour to obtain one estimate of each measure per hour-long period.

Language and acoustic feature extraction. A microphone embedded in each partner's smartphone recorded audio during the study period. The audio clips were 3 minutes long and collected once every 12 minutes, resulting in 6 minutes of audio per 12 minutes per pair (male and female within a couple). This resulted in a reasonable tradeoff between the size of the audio data available for storage and processing and the amount of acquired information. For privacy considerations, participants were instructed to mute their microphones when in the presence of anyone not in the study.

We transcribed and processed audio recording using Linguistic Inquiry and Word Count (LIWC) software. (Pennebaker, 2007; the entire disclosure of which is hereby incorporated by refernce). For our theoretically driven features (task 1), we used preset dictionaries representing personal pronouns (such as “I” and “we”), certainty words (such as “always” and “must”), and negative emotion words (such as “tension” and “mad”). To test unimodal combinations of features, we used four preset LIWC categories, including linguistic factors (25 features including personal pronouns, word count, and verbs), psychological constructs (32 features such as words relating to emotions and thoughts), personal concern categories (seven features such as work, home, and money), and para linguistic variables (three features such as assents and fillers).

Voice-activity detection (VAD) was used to automatically chunk continuous audio streams into segments of speech or nonspeech. We used speaker clustering and gender identification to automatically assign a gender to each speech segment. We then extracted vocally encoded indices of arousal (F0 and intensity). To map the low-level acoustic descriptors onto a vector of fixed dimensionality—independent of the audio clip duration—we further computed the mean, SD, maximum value, and first-order coefficient of the linear regression curve over each speech segment, resulting in eight features. All acoustic and language features were calculated separately by partner and averaged per hour.

Context and interaction indices. In addition to our vocal, language, self-reported, and physiological variables, we assessed numerous other factors that are potentially relevant for identifying conflict episodes. The contextual variables included whether participants consumed caffeine, alcohol, tobacco, or other drugs; whether they were driving; whether they exercised; body temperature; and physical activity level. The interactional variables involved the GPS-based distance between partners and information related to whether the dating partners were together, interacting face to face, or communicating via phone call or text messaging and if they were with other people.

The data for the contextual and interactional feature groups were collected via various mechanisms, including physiological sensors, smartphones, self-reports based on the hourly surveys, and interview data.

Conflict. We identified the hours in which conflicts occurred using the self-report phone surveys. For each hour, participants reported whether they “expressed annoyance or irritation” toward their dating partner using a dichotomous yes/no response option. Because determining what constitutes a conflict is subjective, we elected to use a discrete behavioral indicator (that is, whether the person said something out of irritation) as our ground-truth criterion for determining if conflict behavior occurred within a given hour. This resulted in 53 hours of conflict behavior and 182 hours of no conflict behavior for females and 39 hours of conflict behavior and 206 hours of no conflict behavior for males.

Conflict Classification System

The goal of the classification task was to retroactively distinguish between instances of conflict behavior and no conflict behavior, as reported by the participants. The analysis windows constituted nonoverlapping hourly instances starting at 10:00 a.m. and ending at bedtime.

To classify conflict, we used a binary decision tree because of its efficiency and self-explanatory structure. We employed a leave-one-couple-out cross-validation setup for all classify-cation experiments. For tasks 2 and 3, feature transformation was performed through a deep autoassociative neural network, also called an autoencoder, with three layers in a fully unsupervised way. The autoencoder's bottle neck features at the middle layer consisted of the input of a binary tree for the final decision (Y=conflict and N=no conflict). Unimodal classification followed a similar scheme, under which the autoencoder transformed only the within-modality features. FIG. 4 presents a schematic representation of the classification system as it applies to our dataset.

Further details regarding the system, a list of the entire feature set, complete results from all our experiments, and confusion matrices (that is, tables showing the performance of the classification model) are available online at homedata.github.io/statistical-methodology.html.

Results

Our study results showed that several of our theoretically driven features (such as self-reported levels of anger, HRV, negative emotion words used, and mean audio intensity) were associated with conflict at levels significantly higher than chance, with an unweighted accuracy (UA) reaching up to 69.2 percent for anger and 62.3 percent for expressed negative emotion (task 1). This initial set of results is in line with laboratory research linking physiology and language use to couples' relationship functioning. (Amato, 2000; Levenson and Gottman, 1985; Timmons, Margolin, and Saxbe, 2015; Simmons, Gordon, and Chambless, 2005; Baucom, 2012). When testing unimodal feature groups (task 2), the levels of accuracy reached up to 66.1 and 72.1 percent for the female and male partners, respectively. Combinations of modalities based on EDA, ECG activity, synchrony scores, language used, acoustic data, self-reports, and context and interaction resulted in UAs up to 79.6 percent (sensitivity=73.5 percent and specificity=85.7 percent) for females and 86.8 percent (sensitivity=82.1 percent and specificity=91.5 percent) for males. Using all features except self-reports, the UA reached up to 79.3 percent. These findings generally indicate that it is possible to detect a complex, psychological state with reasonable accuracy using multi-modal data obtained in uncontrolled, real-life settings.

Because we aim to eventually detect conflict using passive technologies only—that is, without requiring couples to complete self-report surveys— we compared the UAs based only on self-reported MQI to combinations incorporating passive technologies (task 4). These results showed several setups where multimodal feature groups with and without self-reported MQI data significantly exceeded the UA achieved from MQI alone. This indicates that the passive technologies added predictability to our modeling schemes.

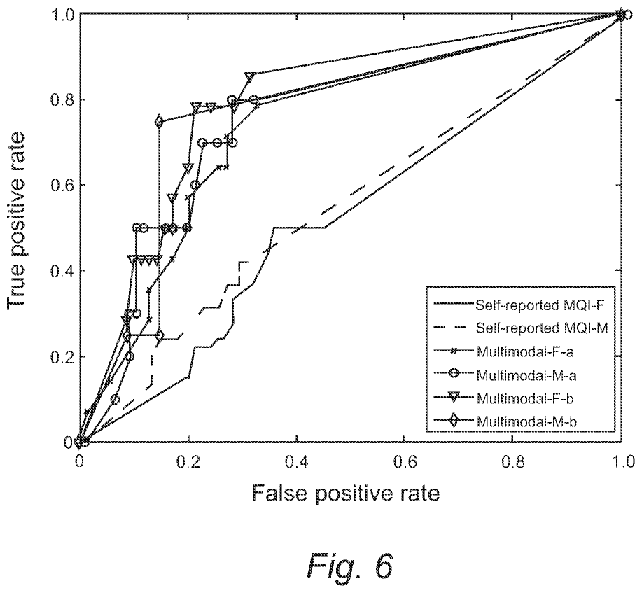

FIG. 6 shows receiver operating characteristic (ROC) curves for several feature combinations. The results showed that the area under the curve (AUC) for our multimodal indices reached up to 0.79 for females and 0.76 for males.

Discussion

The results we report here provide a proof of concept that the data collected via mobile computing methods are valid indicators of interpersonal functioning in daily life. Consistent with laboratory-based research, we found statistically significant above-chance associations between conflict behavior and several theoretically driven, individually tested data features. We also obtained significant associations between conflict and both uni-modal and multimodal feature groups with and without self-reported MQI included. In fact, our best-performing combinations of data features in several cases reached or exceeded the UA levels obtained via self-reported MQI alone.

To our knowledge, the prototype model developed for this study is the first to use machine-learning classification to identify episodes of conflict behavior in daily life using multimodal, passive computing technologies. Our study extends the literature by presenting an initial case study indicating that it is possible to detect complex psychological states using data collected in an uncontrolled environment.

Implications

Couples communicate using complex, cross-person interactional sequences where emotional, physiological, and behavioral states are shared via vocal cues and body language. Multimodal feature detection can provide a comprehensive assessment of these inter-actional sequences by monitoring the way couples react physiologically, what they say to each other, and how they say it. Couples in distressed relation-ships can become locked into maladaptive patterns that escalate quickly and are hard to exit once triggered. Detecting and monitoring these sequences as they occur in real time could make it possible to interrupt, alter, or even pre-vent conflict behaviors.

Thus, although preliminary, our data are an important first step toward using mobile computing methods to improve relationship functioning. The proposed algorithms could be used to identify events or experiences that precede conflict and send prompts that would decrease the likelihood that such events will spill over to affect relationship functioning. Such interventions would move beyond the realm of human-activity recognition to also include the principles of personal informatics, which help people to engage in self-reflection and self-monitoring to increase self-knowledge and improve functioning. For example, a husband who is criticized by his boss at work might experience a spike in stress levels, which could be reflected in his tone of voice, the content of his speech, and his physiological arousal. Based on this individual's pattern of arousal, our system would predict that he is at increased risk for having an argument with his spouse upon returning home that evening. A text message could be sent to prompt him to engage in a meditation exercise, guided by a computer program, that decreases his stress level. When this individual returns home, he might find that his children are arguing and that his wife is in an irritable mood. Although such situations often spark conflict between spouses, the husband might feel emotionally restored following the meditation exercise and thus be able to provide support to his wife and avoid feeling irritable himself, thereby preventing conflict.

A second option is to design prompts that are sent after a conflict episode to help individuals calm down, recover, or initiate positive contact with their partners. For example, a couple living together for the first time might get in an argument about household chores. After the argument is over, a text message could prompt each partner to independently engage in a progressive muscle relaxation exercise to calm down. Once they are in a relaxed state, the program could send a series of prompts that encourage self-reflection and increase insight about the argument—for example, what can I do to communicate more positively with my partner? What do I wish I had done differently?

In addition to detecting conflict episodes, amplifying positive moods or the frequency of positive interactions could be valuable. Potential behavioral prompts could include exercises that build upon the positive aspects of a relationship, such as complimenting or doing something nice for one's partner. Employing these methods in people's daily lives could increase the efficacy of standard therapy techniques and improve both individual and relationship functioning. Because the quality of our relationships with others plays a central role in our emotional functioning, mobile technologies thus provide an exciting approach to promoting well-being.

Limitations

Although the results from our classification experiments suggest that these methods hold promise, our findings should be interpreted in light of several limitations. Our system's classification accuracy, while moderately good given the task's inherent complexity, will need to be improved before our method can be employed widely. In our best-performing models, we missed 18 percent of conflict episodes and falsely identified 9 per-cent of cases as conflict. Classification systems that miss large numbers of conflict episodes will be limited in their ability to influence people's behaviors. At the same time, falsely identifying conflict would force people to respond to unnecessary behavior prompts, which could annoy them or cause them to discontinue use.

In our current model, classification accuracy is inhibited by several factors.

First, we relied on self-reports of conflict. Future projects could use audio recordings as an alternative, perhaps more accurate, way to identify periods of conflict.

Second, conflict and how it is experienced and expressed is highly variable across couples, with people showing different characteristic patterns in physiology or vocal tone. For example, some couples yell loudly during conflict, whereas others with-draw and become silent. One method for addressing this issue could be to train the models on individual couples during an initial trial period. By tailoring our modeling schemes, we might be able to capture response patterns specific to each person and thereby improve our classification scores.

Third, we collapsed our data into hour-long time intervals, which likely caused us to lose important information about when conflict actually started and stopped. Many conflict episodes do not last for an entire hour, and physiological responding within an hour-long period could reflect various activities besides conflict. Using a smaller time interval would likely increase accuracy.

Fourth, outside of the synchrony scores, we did not take into account the joint effects of male and female responses. Considering these together (such as male and female vocal pitch increasing at the same time) could improve our results.

Additional Studies

New algorithms have been developed to expand the types of interpersonal processes that could be detected for different constructs. In particular, an algorithm that could detect when couples were interacting with each other with 99% accuracy (balanced accuracy=0.99, kappa=0.97, sensitivity=0.98, specificity=0.99) was developed. An algorithm was then developed to detect positive mood (real, self-reported mood correlated with predicted mood at 0.75, with a mean absolute error of 14.54 on a 100-point scale). Similarly, an algorithm to detect feelings of closeness between romantic partners (real self-reported feelings of closeness between romantic partners correlated with predicted closeness at 0.85, with a mean absolute error of 11.40 on a 100-point scale). The original algorithm was also improved upon the original algorithm that detected couple conflict (from the first article). The updated algorithm was able to detect when couples were having conflict with 96% accuracy (balanced accuracy=0.96, kappa=0.90, sensitivity=0.92, specificity=0.99). Table 1 provides details of this algorithm:

TABLE 1
Conflict characterization model
Discrete

| | |
|---|---|
| Interacting | Multilayer perceptron neural network |
| Conflict | Classification trees with boosting |

Continuous

| | |
|---|---|
| Positive mood | Regression modeling using rules |
| Closeness | Random forest |

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

### REFERENCES

Amato, P. R. “The Consequences of Divorce for Adults and Children,” J. Marriage and Family, vol. 62, no. 4, 2000, pp. 1269-1287
Baucom B. R. et al., “Correlates and Characteristics of Adolescents' Encoded Emotional Arousal during Family Conflict,” Emotion, vol. 12, no. 6, 2012, pp. 1281-1291.
Burman, B., & Margolin, G. (1992). Analysis of the association between marital relationships and health problems: An interactional perspective. Psychological Bulletin, 112, 39-63. doi: 10.1037/0033-2909.112.1.39
Chaspari, T. et al., “Quantifying EDA Synchrony through Joint Sparse Representation: A Case-Study of Couples' Interactions,” Proc. Int'l Conf. Acoustics, Speech, and Signal Processing (ICASSP 15), 2015, pp. 817-821.
Cicila, L. N., Georgia, E. J., & Doss, B. D. (2014). Incorporating internet-based interventions into couple therapy: Available resources and recommended uses. The Australian and New Zealand Journal of Family Therapy, 35, 414. doi: 10.1002/anzf.1077
Coan, J. A., Schaefer, H. S., & Davidson, R. J. (2006). Lending a hand: Social regulation of the neural response to threat. Psychological Science, 17, 1032-1039. doi: 10.1111/j.1467-9280.2006.01832.x
Doss, B. D., Bensen, L. A., Georgia, E. J., & Christensen, A. (2013). Translation of Integrative Behavioral Couple Therapy to a web-based intervention. Family Process, 52, 139-153. doi: 10.1111/famp.12020
Gideon, J., Provost, E. M., & McInnis, M. (2016). Mood state prediction from speech of varying acoustic quality for individuals with bipolar disorder. Proceedings of the IEEE International Conference on Acoustics, Speech and Signal Processing, 2359-2363.
Grewen, K. M., Andersen, B. J., Girdler, S. S., & Light, K. C. (2003). Warm partner contact is related to lower cardiovascular reactivity. Behavioral Medicine, 29, 123-130. doi: 10.1080/08964280309596065
Hung, H., & Englebienne, G. (2013). Systematic evaluation of social behavior modeling with a single accelerometer. Processing of the ACM International Joint Conference on Pervasive and Ubiquitous Computing, 127-139. doi: 10.1145/2494091.2494130
Holt-Lunstad, J., Smith, T. B., & Layton, J. B. (2010). Social relationships and mortality risk: A meta-analytic review. PLoS Medicine, 7, e1000316. doi: 10.1371/journal.pmed.1000316
Ianakieva, I., Fergus, K., Ahmad, S., Pos, A., & Pereira, A. (2016). A model of engagement promotion in a professionally facilitated online intervention for couples affected by breast cancer. Journal of Marital and Family Therapy, 42, 701-715. doi: 10.1111/jmft.12172
Kim, S., Valente, F., & Vinciarelli, A. (2012). Automatic detection of conflict in spoken conversations: Ratings and analysis of broadcast political debates. Proceedings of IEEE International Conference on Acoustic, Speech, and Signal Processing, 5089-5092. doi: 10.1109/ICASSP.2012.6289065
Lane, N. D., Lin, M., Mohammod, M., Yang, X., Lu, H., Cardone, G., . . . & Choudhury, T. (2014). Bewell: Sensing sleep, physical activities and social interactions to promote wellbeing. Mobile Networks and Applications, 19, 345-359. doi: 10.1007/s11036-013-0484-5
Larson, J. H., Vatter, R. S., Galbraith, R. C., Holman, T. B., & Stahmann, R. F. (2007). The RELATionship Evaluation (RELATE) with therapist-assisted interpretation: Short-term effects on premarital relationships. Journal of Marital and Family Therapy, 33, 364-374. doi: 10.1111/j.1752-0606.2007.00036.x
Lawler, J. (2010). The real cost of workplace conflict. Entrepreneur. Accessed from https://www.entrepreneur.com/article/207196.
Leach, L. S., Butterworth, P., Olesen, S. C., & Mackinnon, A. (2013). Relationship quality and levels of depression and anxiety in a large population-based survey. Social Psychiatry and Psychiatric Epidemiology, 48, 417-425. doi: 10.1007/s00127-012-0559-9
Lee, Y., Min, C., Hwang, C., Lee, J., Hwang, I., Ju, Y., . . . Song J. (2013). SocioPhone: Everyday face-to-face interaction monitoring platform using multi-phone sensor fusion. Proceedings of the International Conference on Mobile Systems, Applications, and Services, 375-388. doi: 10.1145/2462456.2465426
Levenson R. W. and Gottman, J. M., “Physiological and Affective Predictors of Change in Relationship Satisfaction,” J. Personality and Social Psychology, vol. 49, no. 1, 1985, pp. 85-94.
Lunbald, A., & Hansson, K. G. (2005). The effectiveness of couple therapy: Pre- and post-assessment of dyadic adjustment and family climate. Journal of Couple & Relationship Therapy, 4, 39-55. doi: 10.1300/J398v04n04_03
Mayberry, R. M., Nicewander, D. A., Qin, H., & Ballard, D. J. (2006). Improving quality and reducing inequality: A challenge in achieving best care. Proceedings of Baylor University Medical Center, 19, 103-118
Pennebaker, J. W. et al., “The Development and Psychometric Properties of LIWC2007,” 2007; www.liwc.net/LIWC2007LanguageManual.pdf
Robles, T. F., & Kiecolt-Glaser, J. K. (2003). The physiology of marriage: Pathways to health. Physiology and Behavior, 79, 409-416. doi: 10.1016/S0031-9384(03)00160-4
Sacker, A. (2013). Mental health and social relationships. Evidence Briefing of the Economic and Social Research Council. Accessed from http://www.esrc.ac.uk/files/news-events-and-publications/evidence-briefings/mental-health-and-social-relationships/
Simmons, R. A., Gordon, P. C., and Chambless, D. L., “Pronouns in Marital Interaction: What Do ‘You’ and ‘I’ Say about Marital Health?,” Psychological Science, vol. 16, no. 12, 2005, pp. 932-936
Sonnentag, S., Unger, D., & Nagel, I. (2013). Workplace conflict and employee well-being: The moderating role of detachment from work during off-job time. International Journal of Conflict Management, 24, 166-183. doi: 10.1108/10444061311316780
Springer, K. W., Sheridan, J., Kuo, D., & Carnes, M. (2007). Long-term physical and mental health consequences of childhood physical abuse: Results from a large population-based sample of men and women. Child Abuse & Neglect, 31, 517-530. doi: 10.1016/j.chiabu.2007.01.003
Timmons, A. C., Chaspari, T., Han, S. C., Perrone, L., Narayanan, S., & Margolin, G. (2017). Using multimodal wearable technology to detect conflict among couples. IEEE Computer, 50, 50-59. doi:10.1109/MC.2017.83
Timmons, A. C. Margolin, G. and Saxbe, D. E., “Physiological Linkage in Couples and Its Implications for Individual and Interpersonal Functioning,” J. Family Psychology, vol. 29, no. 5, 2015, pp. 720-731

What is claimed is:

1. A method comprising:
receiving data streams from a plurality of mobile smart devices in possession of a plurality of users, the data streams recording information about users' daily lives, the data streams generated by the mobile smart devices or sensors in communication with the mobile smart devices; and
sending intervention signals to a user in response to data acquired from two or more individuals and interpreted with respect to user internal states, moods, emotions, predetermined behaviors, and interactions with other users.

2. The method of claim 1 wherein the data streams are classified or quantified into classifications or quantifications.

3. The method of claim 1 wherein signal-derived features are extracted from the data streams, the signal-derived features providing inputs to a trained neural network that determined interpersonal classifications that allow selection of a predetermined intervention to be sent.

4. The method of claim 1 wherein the intervention signals are determined by algorithmic signal processing and/or machine learning solutions such that the intervention signals are responsive, interactive, and adaptive to the users.

5. The method of claim 4 further comprising incorporating human expert knowledge into a determination of the intervention signals.

6. The method of claim 5 wherein human expert knowledge is integrated and includes prompts sent at random intervals and/or according to specific time schedules.

7. The method of claim 6 wherein reminders designed to help users reach their daily goals are sent.

8. The method of claim 7 wherein the reminders include as spending a certain amount of time together, achieving a certain ratio of positive to negative interactions, or having a certain amount of physical contact.

9. The method of claim 1 wherein sending of interventions triggered by algorithms that automatically detect and predict moods and events to send prompts to oneself or to other users in a social network.

10. The method of claim 9 wherein moods and events include risky behaviors, extreme emotions, and/or negative moods.

11. The method of claim 9 wherein the prompts include warning people that conflict or other events are likely to occur, prompting people to engage in relaxation exercises, take a break, give a compliment, or to do something nice for someone else.

12. The method of claim 9 wherein the interventions also include sending prompts after events of interest have occurred.

13. The method of claim 9 wherein the prompts instruct users to reflect on an occurrence of an event, engage in relationship building activities, initiate positive contact, or discuss a topic together.

14. The method claim 1 further comprises providing feedback to the users to encourage beneficial aspects of interpersonal relationships.

15. The method of claim 14 wherein expert-knowledge is applied with personal and interpersonal information captured from human monitoring systems integrated through signal processing, data-scientific, and machine learning solutions.

16. The method of claim 15 wherein a human state is recognized, understood, and predicted and actionable feedback is provided to improve it in relation to corresponding relationship functioning.

17. The method of claim 14 wherein measurable indices of individual and interpersonal behavior consisting of input for closed-loop systems that automatically provide suggestions towards a desired state.

18. The method of claim 1 wherein heuristic, machine-learning, or control-theoretical approaches are applied and are automatically trained/tuned/perturbed towards optimizing a desired criterion to minimize conflict and maximize positive interactions.

19. The method of claim 1 wherein a model is constructed for interpersonal dynamics that occur when a set of individuals linked through a relationship interact with each other and with their environment.

20. The method of claim 1 further comprising learning each user's patterns over time so that accuracy and effectiveness of interventions increase with use.

21. The method of claim 1 further comprising investigating an impact of each prompt and intervention on individual and interpersonal functioning and providing feedback about which interventions are most helpful population-wide and which are better for specific users or groups of users.

22. The method of claim 1 wherein intervention schemes are performed quantitatively through signal- and data-derived measures indicative of individual characteristics and relationship functioning concepts.

23. A system comprising a plurality of mobile smart devices operated by a plurality of users wherein at least one smart device or a combination of smart devices execute steps of:
receiving data streams from a plurality of mobile smart devices in possession of a plurality of users, the data streams recording information about users' daily lives; and
sending intervention signals to a user in response to data acquired from two or more individuals and interpreted with respect to user internal states, moods, emotions, predetermined behaviors, and interactions with other users.

24. The system of claim 23 further comprising a plurality of sensors worn by the plurality of users.

25. The system of claim 23 wherein the mobile smart devices include a microprocessor and non-volatile memory on which instructions for implementing the steps are stored.
